(19) United States
(12) Patent Application Publication
(10) Pub. No.: US 2025/0120831 A1
(43) Pub. Date: Apr. 17, 2025

(54) INTELLIGENT WARD ROUND METHOD AND SYSTEM BASED ON SSVEP ELECTROENCEPHALOGRAM SIGNAL

(71) Applicant: ANHUI UNIVERSITY, Hefei (CN)

(72) Inventors: Zhao LV, Hefei (CN); Cunhang FAN, Hefei (CN); Ping LI, Hefei (CN); Huabin WANG, Hefei (CN); Lei ZHANG, Hefei (CN); Chao ZHANG, Hefei (CN); Xinhui LI, Hefei (CN); Shulin DAI, Hefei (CN); Chang ZHOU, Hefei (CN); Zitong SUN, Hefei (CN)

(21) Appl. No.: 19/002,641

(22) Filed: Dec. 26, 2024

Related U.S. Application Data
(63) Continuation of application No. PCT/CN2023/136419, filed on Dec. 5, 2023.

(51) Int. Cl. A61F 4/00; A61B 5/00; A61B 5/374; A61B 5/378; G06F 3/01

(52) U.S. Cl. CPC A61F 4/00; A61B 5/374; A61B 5/378; A61B 5/7246; A61B 5/725; G06F 3/015

(57) ABSTRACT

The present invention discloses an intelligent ward round method and system based on an SSVEP electroencephalogram signal, the method including: the interactive terminal is used to present a visual stimulus source that includes a plurality of candidate intention options to the patient, the acquisition terminal is used to collect the patient's electroencephalogram signal, the electroencephalogram signal obtained by the acquisition terminal is transmitted to the interactive terminal for electroencephalogram signal recognition, the patient's expression intention is determined, and the expression intentions comprise disease intentions, privacy intentions, psychological intentions, physiological intentions, environmental intentions, and safety intentions, and the intelligent ward round is completed. This method greatly improving the ability of communication between the patient and medical staff.

### CROSS-REFERENCE TO RELATED APPLICATIONS

This application is a continuation of International Patent Application No. PCT/CN2023/136419, filed on Dec. 5, 2023. The content of the aforementioned applications, including any intervening amendments thereto, are incorporated herein by reference.

### TECHNICAL FIELD

The present invention relates to the field of brain-computer interface technology, and in particular, to an intelligent ward round method and system based on an SSVEP electroencephalogram signal.

### BACKGROUND

Research shows that ⅓ of stroke patients have aphasia, and these patients lose both a four-limb movement ability and a communication ability, and thus cannot effectively communicate with medical staff to express own needs. At present, a brain-computer interface technique is continuously developed, and an application of the brain-computer interface technique to the intelligent medical field becomes a trend. Steady-state visual evoked potential (SSVEP) has the advantages of simple design, a stable signal, a small number of leads, a high transmission rate, or the like, and an intention of the patient is identified through an SSVEP electroencephalogram signal to assist the patient in communicating with the medical staff, thus greatly improving the ability of telling the needs of the patient.

However, due to complex characteristics of the electroencephalogram signal, a large number of noise signals of irrelevant brain activities and artifacts exist in the collected SSVEP electroencephalogram signal, and part of noise has relevance to stimulus frequency recognition, such as ocular artifacts, or the like, so that a signal-to-noise ratio of the SSVEP electroencephalogram signal is quite low, and frequency recognition is seriously obstructed.

In the prior art, for example, empirical modal decomposition and wavelet transformation are adopted to decompose and denoise the electroencephalogram signal, but both the empirical modal decomposition and the wavelet transformation have certain limitation; for example, modal aliasing is prone to occur in the empirical modal decomposition. Secondly, in these decomposition methods, only the noise signals are concerned, measurement of boundaries and relationships between the noise signals and valid signals is not considered, and the calculated “noise” is directly filtered out, causing feature loss of different degrees of the original electroencephalogram signal.

### SUMMARY

In view of the problems existing in the prior art, the present invention provides an intelligent ward round method and system based on an SSVEP electroencephalogram signal, which aim at guaranteeing a high signal-to-noise ratio and high recognition rate of the SSVEP electroencephalogram signal and meanwhile giving consideration to a real-time performance, so as to ensure that a patient can communicate with medical staff without obstacles to complete an intelligent ward round requirement.

In order to achieve the above purpose, the present invention provides the following technical solution.

In a first aspect, the present invention provides an intelligent ward round method based on an SSVEP electroencephalogram signal, including:
acquiring a preprocessed electroencephalogram signal of a patient under a stimulus of a current visual stimulus source, recording the preprocessed electroencephalogram signal as a first electroencephalogram signal, and defining a reference signal according to a visual stimulus frequency for evoking the first electroencephalogram signal;
decomposing the first electroencephalogram signal into sub-band components distributed in different frequency ranges, calculating differences of the sub-band components, a difference of the reference signal and differences between the sub-band components and the reference signal, and acquiring a total spatial filter of the sub-band components;
processing each sub-band component by the total spatial filter, rearranging the sub-band components into a new multi-channel signal, and recording the new multi-channel signal as a second electroencephalogram signal;
acquiring a plurality of variation modal components of the second electroencephalogram signal by adopting variation modal decomposition, and optimizing weights of the variation modal components under each channel to reconstruct an electroencephalogram signal to obtain a third electroencephalogram signal; and
obtaining an evoking stimulus frequency of the third electroencephalogram signal based on a maximum correlation coefficient of a correlation coefficient of the third electroencephalogram signal and a correlation coefficient of the third electroencephalogram signal and the reference signal, and determining an intention of the patient based on the evoking stimulus frequency to complete an intelligent ward round.

In a second aspect, the present invention provides an intelligent ward round device based on an SSVEP electroencephalogram signal, including:
an SSVEP signal collecting module configured to collect a first electroencephalogram signal of a patient under a stimulus of a current visual stimulus source;
a reference signal defining module configured to define a reference signal according to a visual stimulus frequency for evoking the first electroencephalogram signal;
a sub-band decomposing module configured to decompose the first electroencephalogram signal into sub-band components distributed in different frequency ranges;
a sub-band filtering module configured to calculate differences of the sub-band components, a difference of the reference signal and differences between the sub-band components and the reference signal, and acquire a total spatial filter of the sub-band components to filter the sub-band components;
a signal arranging module configured to rearrange the sub-band components processed by the total spatial filter into a second electroencephalogram signal;
a signal decomposing and reconstructing module configured to acquire a plurality of variation modal components of the second electroencephalogram signal according to variation modal decomposition, and optimize weights of the variation modal components under each channel to reconstruct an electroencephalogram signal to obtain a third electroencephalogram signal; and
a stimulus frequency determining module configured to obtain an evoking stimulus frequency of the third electroencephalogram signal for determining an intention of the patient during an intelligent ward round based on a maximum correlation coefficient of a correlation coefficient of the third electroencephalogram signal and a correlation coefficient of the third electroencephalogram signal and the reference signal.

The intelligent ward round method and system based on an SSVEP electroencephalogram signal according to the present invention have the following beneficial effects.
1. In the present invention, the original SSVEP electroencephalogram signal is subjected to TRCA filtering and feature rearrangement to form the new multi-channel signal, the signal is then decomposed into the plurality of variation modal components by adopting the variation modal decomposition, parameters of the weights of the variation modal components are optimized using a sparrow search algorithm, typical correlation analysis is performed on the electroencephalogram signal after weighted reconstruction and an average signal as well as on the electroencephalogram signal and sine and cosine reference signals, different correlation coefficients are calculated, the highest correlation coefficient is selected as a final recognition target, and a frequency recognition result of the SSVEP electroencephalogram signal is obtained. With multiple times of decomposition and reconstruction of the signal, a valid part of the signal is enhanced, an influence of a noise artifact part in the SSVEP electroencephalogram signal is reduced to the greatest extent, and the extracted SSVEP electroencephalogram signal has a higher signal-to-noise ratio. In the correlation coefficient calculation process, correlation of average features and correlation of the reference signal are calculated, influences of different individuals and trials are considered, and accuracy of frequency recognition is further ensured on the premise of a high signal-to-noise ratio. Then, the intention of the patient is identified through the SSVEP electroencephalogram signal, and the patient is helped to communicate with the medical staff, thus greatly improving the ability of telling needs of the patient.
2. Considering that the SSVEP electroencephalogram signal is weak and susceptible to other electrical signals with correlation with stimulus frequency recognition, in the present invention, the problem that task-related components extracted from the SSVEP electroencephalogram signal by TRCA may be mixed with task-related component noise in the prior art is solved by maximizing the differences of the sub-band components, the difference of the reference signal and the differences between the sub-band components and the reference signal, and the noise is filtered out in each sub-band component, instead of filtering the whole noise of the first electroencephalogram signal, so that an influence between the sub-band components is reduced, undesired frequency information is not prone to introduction, the correlation of visual stimulus tasks is more carefully maximized, and the signal-to-noise ratio of the signal is further improved.
3. In the present invention, the signal is decomposed into the plurality of variation modal components for processing by the variation modal decomposition, thereby reducing an influence of artifacts to the greatest extent. Firstly, a number of the variation modal components is determined through singular values, so as to separate the noise and the valid signals in the variation modal components after the decomposition as much as possible, the variation modal components obtained by the decomposition are weighted by adopting the sparrow search algorithm, and valid components are endowed with larger weights, thus effectively screening noise signals, and improving frequency recognition precision. The number of the better variation modal components is preliminarily determined to guarantee the real-time performance of frequency recognition of the SSVEP electroencephalogram signal, a difference degree between the variation modal components and the original signal is then measured, and the weight of each variation modal component is automatically optimized by adopting the sparrow search algorithm, thus solving the problem that residual components of variation modal decomposition are directly removed by adopting a preset standard in the prior art to possibly influence the valid electroencephalogram signal to different degrees, reproducing relationships of different channels and different frequency signals in the original signal as far as possible while reducing a noise influence of the SSVEP electroencephalogram signal, and improving the accuracy of the frequency recognition.

### BRIEF DESCRIPTION OF DRAWINGS

FIG. 1 is a schematic flow diagram of an intelligent ward round method based on an SSVEP electroencephalogram signal according to the present invention;

FIG. 2 is a schematic flow diagram of decomposing and optimizing a weight under each channel to reconstruct a third electroencephalogram signal in the present invention;

FIG. 3 is a schematic diagram of an overall framework of an intelligent ward round process based on the SSVEP electroencephalogram signal in the present invention; and

FIG. 4 is a structural block diagram of an intelligent ward round system based on an SSVEP electroencephalogram signal according to the present invention;

FIG. 5 is a schematic diagram of the overall structure of an intelligent ward round system based on SSVEP electroencephalogram signals provided by an embodiment of the present invention;

FIG. 6 is a schematic diagram of the main interface of the intelligent ward round system on the interactive terminal provided by an embodiment of the present invention;

Wherein, 101. Interactive terminal; 102. Collection terminal; 103. Display; 105. Processor; 107. Memory; 104. Electrode cap; 106. Amplifier.

### DESCRIPTION OF EMBODIMENTS

The technical solutions in the embodiments of the present invention are clearly and completely described below with reference to the accompanying drawings in the embodiments of the present invention, and apparently, the described embodiments are not all but only a part of the embodiments of the present invention. All other embodiments obtained by a person of ordinary skill in the art based on the embodiments of the present invention without creative efforts shall fall within the protection scope of the present invention.

The present invention applies BCI (brain-computer interface) technology to the traditional ward, by reconstructing the SSVEP electroencephalogram signal with high signal-to-noise ratio, identifies the patient's intention, helps the patient communicate with the medical staff, makes the aphasia patient can clearly express the current needs to the medical staff, assists the medical staff to complete the ward round process, thereby reducing the difficulty and time cost of communication between the doctor and the patient, and getting the feedback of the patient's physical and mental feelings in time is also conducive to the continuation of the next treatment to early recovery.

Among them, SSVEP (steady-state visual evoked potential) refers to the fact that when a fixed frequency of visual stimulation is received, the visual cortex of the human brain will produce a continuous response related to the stimulus frequency. SSVEP has the law of rhythm assimilation, so the frequency of SSVEP induced by different target stimulus frequencies is also different from each other, which makes it possible to identify the brain's thinking activity by analyzing SSVEP, so that it can be used as instructions that can be distinguished from each other.

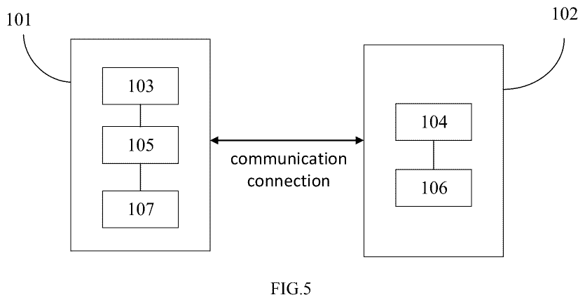

Specifically, the present invention provides an intelligent ward round system based on SSVEP electroencephalogram signals, as shown in FIG. 5, and comprises the interactive terminal 101 and the collection terminal 102. Wherein the interactive terminal 101 may, but is not limited to, comprise the display 103, the processor 105 and the memory 107. Optionally, the interactive terminal 101 can be a device such as a smartphone, a tablet, a laptop, or a personal computer. The acquisition terminal 102 may, but is not limited to, comprise the electrode cap 104 and the amplifier 106. The interactive terminal 101 and the collection terminal 102 can communicate and transmit data through wired or wireless mode.

The memory 107 of the interactive terminal 101 stores a visual stimulus source with a plurality of candidate patient intention options, i.e., a light source that flickers at a specific frequency; This visual stimulus is presented to the patient through the display 103. The patient wears the electrode cap 104 in the acquisition terminal 102, obtains a weak electrical signal on the surface of the scalp through the electrode sheet, and enhances the signal strength through the amplifier 106. The interactive terminal 101 receives the SSVEP signal obtained by the acquisition terminal 102, analyzes and identifies the original SSVEP signal through the processor 105, obtains the patient's current expression intention, and conveys it to the medical staff through the display 103.

The expression intention in this embodiment comprises six first-order intentions, which are:

Illness intentions, including second-level intentions such as surgery and treatment options;

Privacy intentions, including second-level intentions such as going to the toilet, getting dressed, covering a quilt, pulling curtains, going to the toilet, operating with the same gender, and wanting to look at a mobile phone;

Psychological intentions, including second-level intentions such as worrying and wanting family members to accompany them;

Physiological intentions, including second-level intentions such as feeling hot and cold, pain, and wanting to move;

Environmental intentions, including second-level intentions such as noise, light, and time dislocation;

Safety intentions, including second-level intentions such as fear.

As a specific embodiment, the patient wears the electrode cap, the medical staff sets the electrodes according to the 10/20 electrode system, the reference electrode is located in the left earlobe A1, the right earlobe A2, the grounding electrode is located in the brain forehead Gnd, Oz, O1, O2, P0z, P03, P04, P05 and P06 A total of eight electrodes are used to record the SSVEP signal, and the SSVEP signal is sampled at a frequency of 1000 Hz.

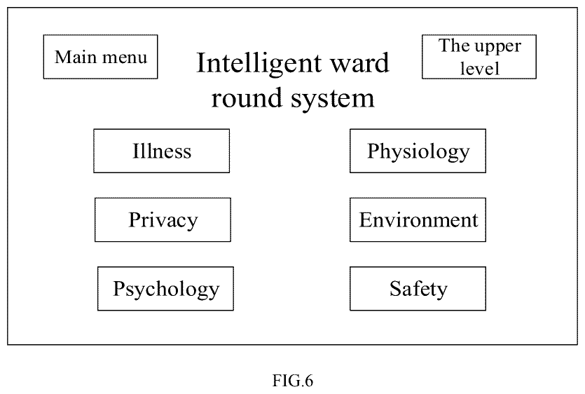

Afterwards, the medical staff uses the tablet computer as an interactive terminal 101 to present the interface of the intelligent ward round system in front of the patient, see FIG. 6, and the intelligent ward round system comprises the main interface and the second-level interface. Among them, the main interface includes six first-level intention scintillation stimulus blocks representing the illness class, privacy class, psychological class, physiological class, environment class and safety class, and two scintillation stimulus blocks representing the main menu and the upper level menu; The second-level interface is a more detailed six-level intention stimulus block corresponding to the six choices on the main interface, and includes two flickering stimulus blocks representing the main menu and the previous menu. exemplary, the frequencies of the six first-level intention scintillation stimulus blocks are 9-14 Hz in turn, and the frequencies of the two scintillation stimulus blocks representing the menu are 10.5 Hz and 11.5 Hz.

The subject's retina was stimulated by displaying the above six different frequencies of scintillation stimulation blocks on the main interface. When the patient looks at one of the flashing light sources, the cerebral cortex produces an electrophysiological response synchronized with the stimulation frequency, and this response usually produces distinct peaks and troughs at the electrode position recorded on the scalp, which is equivalent to the patient choosing one of the six intentions described above, the electrode cap acquires the SSVEP signal and amplifies it, and then transmits the acquired signal to the tablet, the tablet computer stores the SSVEP signal and analyzes the signal through a stored computer program of an intelligent ward round method based on an SSVEP electroencephalogram signal to identify the patient's corresponding first-level intention, and simultaneously produces corresponding display information on the screen of the tablet computer. After the first-level intention recognition, the second-level interface is triggered, and the process of “stimulus-acquisition-analysis-recognition” is repeated at the second-level interface, and finally the patient's specific second-level intention is recognized.

When the analysis result of the processor is the main menu or the upper menu, the corresponding display information will also be generated, and the main menu interface or the upper menu interface will be entered. In the secondary interface, a return unit is also arranged for generating a display control command for the stimulus guidance interface, so that the stimulus guidance interface displays the main interface or the upper level interface.

To facilitate understanding of the present embodiment, an intelligent ward round method based on an SSVEP electroencephalogram signal according to an embodiment of the present invention is described in detail.

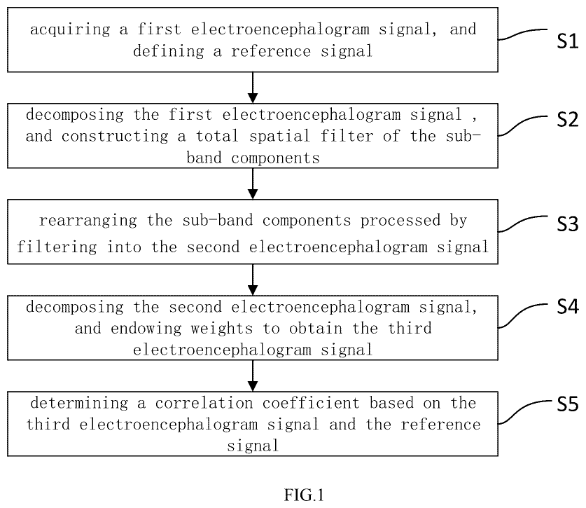

The embodiment of the present invention provides an intelligent ward round method based on an SSVEP electroencephalogram signal, as shown in FIG. 1, including:
S1: acquiring a preprocessed electroencephalogram signal of a patient under a stimulus of a current visual stimulus source, recording the preprocessed electroencephalogram signal as a first electroencephalogram signal, and defining a reference signal according to a visual stimulus frequency for evoking the first electroencephalogram signal;
S2: decomposing the first electroencephalogram signal into sub-band components distributed in different frequency ranges, calculating differences of the sub-band components, a difference of the reference signal and differences between the sub-band components and the reference signal, and acquiring a total spatial filter of the sub-band components;
S3: processing each sub-band component by the total spatial filter, rearranging the sub-band components into a new multi-channel signal, and recording the new multi-channel signal as a second electroencephalogram signal;
S4: acquiring a plurality of variation modal components of the second electroencephalogram signal by adopting variation modal decomposition, and optimizing weights of the variation modal components under each channel to reconstruct an electroencephalogram signal to obtain a third electroencephalogram signal; and
S5: determining a correlation coefficient based on own features of the third electroencephalogram signal and the reference signal to obtain an evoking stimulus frequency of the third electroencephalogram signal, and determining an intention of the patient based on the evoking stimulus frequency to complete an intelligent ward round.

The electroencephalogram signal under the stimulus of the visual stimulus source is an electroencephalogram signal of the patient under a rhythmic visual stimulus in an SSVEP paradigm. For convenience of description, the initially acquired electroencephalogram signal of the patient under the rhythmic visual stimulus in the SSVEP paradigm is hereinafter referred to as an SSVEP signal.

The step S1 includes two aspects: preprocessing and defining the reference signal.Specifically,

S11: preprocessing the acquired SSVEP signal, connecting an electrode with input of a signal collecting module to acquire the SSVEP signal, and outputting the first electroencephalogram signal after amplification, filtering and analog-to-digital conversion. Specifically,

S111: amplifying the SSVEP signal.

Since the SSVEP signal has a quite weak amplitude which typically does not exceed 100 μV, the SSVEP signal is first required to pass through a signal amplification circuit for amplifying the SSVEP signal.

Power supply noise interference inevitably exists when the SSVEP signal is collected, and the SSVEP signal with a required specific frequency is submerged, so that a 50 Hz notch filter is indispensable.

S112: performing power frequency filtering: filtering the SSVEP signal through the 50 Hz notch filter to remove a power frequency.

$$H ( z ) = ( z - {z}_{0} ) / ( z - {z}_{0}^{*} )$$

H(z) is a transfer function of the notch filter, and z₀ and z₀ are conjugate poles on a complex plane and related to a desired 50 Hz frequency.

S113: performing frequency domain filtering: passing the SSVEP signal through a Butterworth filter to obtain the SSVEP signal at the particular frequency (3-40 Hz) required in the present invention.

S114: performing analog-to-digital conversion: converting the collected SSVEP signal from an analog signal into a digital signal for subsequent analysis and processing.

S12: defining sine and cosine periodic signals as the reference signal Y_{f} based on the visual stimulus frequency fᵢ (i=1, 2, . . . , N_{f}) evoking the first electroencephalogram signal, a formula being as follows:

$${Y}_{f} = [ \begin{matrix}sin ( 2 π {f}_{i} t ) \\ cos ( 2 π {f}_{i} t ) \\ … \\ sin ( 2 π {N}_{h} {f}_{i} t ) \\ cos ( 2 π {N}_{h} {f}_{i} t )\end{matrix} ] , t = \frac{1}{{f}_{s}} , \frac{2}{{f}_{s}} , … , \frac{{N}_{s}}{{f}_{s}}$$

wherein Nₕ represents a number of harmonic waves of the stimulus frequency for evoking the first electroencephalogram signal, Nₛ represents a number of channels, fₛ represents a sampling frequency, and N_{f} represents a total stimulus number.

The obtaining the sub-band components of the first electroencephalogram signal in the step S2 includes:

S21: determining a number K of the sub-band components based on a frequency range of the first electroencephalogram signal, a decomposition filter bank including a plurality of band-pass filters, and each band-pass filter being configured to extract information of one specific frequency range of the first electroencephalogram signal to obtain one sub-band component.

Specifically, a frequency range [f₁, f₂] of the first electroencephalogram signal is acquired, wherein f₁ and f₂ represent cut-off frequencies of a low frequency and a high frequency of the first electroencephalogram signal respectively, and in the present embodiment, f₁=3 Hz, f₂=40 Hz, and the number of the sub-band components, i.e., a number of the band-pass filters is determined based on the frequency range and a frequency step length of the first electroencephalogram signal. In the present embodiment, 4 Hz is adopted as the frequency step length; that is, one sub-band component is extracted every 4 Hz.

S22: calculating a center frequency of each band-pass filter, the center frequency being selected based on an equidistant frequency between the frequency ranges of the first electroencephalogram signal.

In the present embodiment, the center frequency is recorded as f_{c}, and f_{c} is taken as the equidistant frequency between f₁ and f₂, so as to cover the entire frequency range of the first electroencephalogram signal.

S23: establishing one band-pass filter for each center frequency, the transfer function of the band-pass filter being:

$$H ( z ) = \frac{z - \frac{1}{2} cos ( 2 π {f}_{c} )}{z - cos ( 2 π {f}_{c} )}$$

wherein f_{c} denotes the center frequency, and z denotes a complex variable.

S24: decomposing the first electroencephalogram signal into K sections in a frequency domain through the decomposition filter bank, each section being one sub-band component to obtain K sub-band components of the first electroencephalogram signal, and the sub-band components being distributed in different frequency ranges.

By establishing the plurality of discrete band-pass filters, the first electroencephalogram signal only keeps a frequency near the specific center frequency of each band-pass filter after passing through the band-pass filter, so as to obtain a signal feature (i.e., the sub-band component) in each frequency section, each sub-band component only contains a feature of a small section of the first electroencephalogram signal, valid features contained in the sections are more similar, and the noise is more obvious. Each section is used for extracting a difference index subsequently, and a noise section irrelevant to an SSVEP stimulus can be more precisely and carefully removed by searching correlation between the signals in the small frequency range sections.

The step S2 of calculating differences of the sub-band components, a difference of the reference signal and differences between the sub-band components and the reference signal, and acquiring a total spatial filter of the sub-band components specifically includes:
maximizing a trial difference index of the same sub-band component in different trials under the same stimulus, a reference difference index between the same sub-band component and the reference signal in different trials under the same stimulus and a template difference index of the reference signal by using a TRCA method, acquiring the spatial filter of the sub-band component under the corresponding stimulus, and connecting all the spatial filters under each stimulus to obtain the total spatial filter of each sub-band.

The trial difference index of the same sub-band component in different trials under the same stimulus is the difference of the sub-band component under the same stimulus, the reference difference index between the same sub-band component and the reference signal in each trial under the same stimulus is the difference between the sub-band component and the reference signal under the same stimulus, and the template difference index of the reference signal is the difference between the sine and the cosine of the reference signal.

The trial difference index, the reference difference index and the template difference index are measured through covariances, and specifically:

the trial difference index is a sum of the covariances of the same sub-band component in different trials under the same stimulus and is recorded as

S₁=Σ_{i,j} Cov(x_{K}ⁱ,X_{K}^{j})

wherein i and j represent different trials under the same stimulus, i≠j, K represents a Kth sub-band component, Nₜ represents a number of the trials, and Cov ( ) represents the covariance;

the reference difference index includes a sum S₂₁ of covariances between the same sub-band component in the trials under the same stimulus and a sine periodic signal in the reference signal and a sum S₂₂ of covariances between the same sub-band component in the trials under the same stimulus and a cosine periodic signal in the reference signal;

wherein

S₂₁=Σᵢ^{Nt} Cov(x_{K}ⁱ,Y_{f}^{s}),

and Y_{f}^{c} is the sine periodic signal in the reference signal;

S₂₂=Σᵢ^{Nt} Cov(x_{K}ⁱ,Y_{f}^{c}),

and Y_{f}^{c} is the cosine periodic signal in the reference signal;

X_{K}ⁱ represents a sub-component of the Kth sub-band component at an ith trial;

the template difference index is a sum of covariances between the sine and cosine periodic signals,

the template difference index is recorded as

S₃=Σᵢ^{Nt} Cov(Y_{f}^{s},Y_{f}^{c}),

wherein Y_{f}^{s} and Y_{f}^{c} are the sine periodic signal and the cosine periodic signal in the reference signal.

The obtaining a total spatial filter of the sub-bands specifically includes:

constructing a difference matrix

$$S = [ \begin{matrix}{S}_{1} & {S}_{21} \\ {S}_{22} & {S}_{3}\end{matrix} ]$$

based on the trial difference index, the reference difference index and the template difference index;

solving

Wₙ^{K}=argmax(Wₙ^{K})^{T}Sₙ^{K}Wₙ^{K}

to obtain a weight of the spatial filter, and constructing the spatial filter under the corresponding stimulus; wherein Wₙ^{K} is the weight of the spatial filter, and Sₙ^{K} is the difference matrix of the Kth sub-band component under the nth stimulus; and

obtaining the final spatial filter for the K sub-band components by connecting the weights of all the spatial filters at each stimulus:

W^{K}=[W₁^{K},W₂^{K}, . . . ,W_{N}_{_{f}}^{K}],

wherein N_{f} represents a total number of the stimuli.

It is considered that some other electrical signals having task correlation, such as ocular electrical noise, inevitably exist in the SSVEP signal, and may have frequency overlaps with the SSVEP signal; in brief, the ocular electrical noise generated under the rhythmic visual stimulus in the SSVEP paradigm is also related to the SSVEP stimulus due to a normal physiological activity of the human body, and the ocular electrical noise cannot be removed by frequency filtering. In the present invention, the problem that task-related components extracted from the SSVEP signal by TRCA may be mixed with task-related component noise in the prior art is solved by maximizing the differences of the sub-band components, the difference of the reference signal and the differences between the sub-band components and the reference signal, the noise is filtered out in each sub-band component, instead of filtering the whole noise of the first electroencephalogram signal, and therefore, the correlation of visual stimulus tasks is more carefully maximized, and the signal-to-noise ratio of the first electroencephalogram signal is further improved.

The step S3 specifically includes:
vertically arranging and organizing the first electroencephalogram signal subjected to TRCA space domain filtering into new channels along the y axis according to a sequence of the N_{f} spatial filters; and
recombining the K sub-band components along the z axis in each new channel to obtain a new signal formed by rearrangement, and recording the new signal as the second electroencephalogram signal.

For N_{f} stimulus targets, N_{f} space domain filters can be obtained, and filtered signals processed by different space domain filters have complementary information of the first electroencephalogram signal, just like signals collected by electrodes at different scalp positions. Therefore, the first electroencephalogram signals processed by different spatial filters are arranged into a vertical column along the y axis, each row represents one first electroencephalogram signal processed by one spatial filter, and then, the K sub-band components of the first electroencephalogram signal are arranged in each row along the z axis to obtain the second electroencephalogram signal. Therefore, each row of the z axis represents one first electroencephalogram signal, and each column of the y axis represents one sub-band component, so that the complementary information of the SSVEP signal can be combined conveniently; for each sub-band component, an influence of the noise is filtered out as far as possible after the above difference maximizing method.

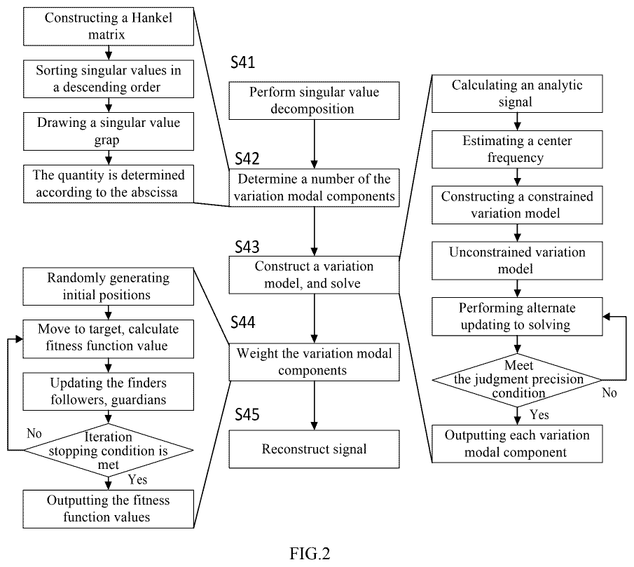

Referring to FIG. 2, the step S4 specifically includes:

S41: representing the second electroencephalogram signal as a sum of the plurality of variation modal components to carry out singular value decomposition on the second electroencephalogram signal.

An ith modal component of the channel Nₛ is

uᵢ^{Ns}(t)=Aᵢ^{Ns}(t)cos(øᵢ^{Ns}(t)),

Aᵢ^{Ns}(t) represents an instantaneous amplitude of the ith modal component of the channel Nₛ, and øᵢ^{Ns}(t) represents an instantaneous phase function of the ith modal component of the channel Nₛ; then, the second electroencephalogram signal is represented as

$$X = {Σ}_{i = 1}^{{N}_{s}} x ( {N}_{s} ) = {Σ}_{i = 1}^{{N}_{s}} {Σ}_{i = 1}^{P} {A}_{i}^{{N}_{s}} ( t ) cos ( {∅}_{i}^{{N}_{s}} ( t ) ) .$$

S42: determining a number P of the variation modal components based on a variation trend of singular values.

S43: constructing a variation model based on the number P of the variation modal components, and solving the variation model by adopting an alternating direction multiplier algorithm to acquire the plurality of variation modal components of the second electroencephalogram signal.

The variation modal component contains local information features of the original signal at different time scales, and can stabilize non-stationary SSVEP signal data. Meanwhile, the variational modal decomposition avoids a modal aliasing problem of empirical modal decomposition.

S44: weighting the variation modal components of each frequency band by adopting a sparrow search algorithm, weights of the variation modal components being determined according to a fitness value of the sparrow search algorithm.

S45: reconstructing the electroencephalogram signal under each channel based on the weights of the variation modal components to obtain the third

The S42 specifically includes:
S421: constructing an m×n order Hankel matrix based on the variation modal components and the channel number of the second electroencephalogram signal,
wherein

$$m = [ \frac{{N}_{s}}{2} + 1 ] , n = {N}_{s} - m + 1 ,$$

Nₛ is the channel number, and [ ] represents upward rounding.

The Hankel matrix obtained according to the second electroencephalogram signal

X=Σᵢ₌₁^{Ns}X(Nₛ)=Σᵢ₌₁^{Ns}Σᵢ₌₁^{Ns}Σᵢ₌₁^{P}Aᵢ^{Ns}(t)Cos(øᵢ^{Ns}(t))

and the channel number Nₛ is:

$$H = [ \begin{matrix}X ( 1 ) & X ( 2 ) & … & X ( {N}_{s} - m + 1 ) \\ X ( 2 ) & X ( 3 ) & … & X ( {N}_{s} - m + 2 ) \\ ⋮ & ⋮ & & ⋮ \\ X ( m ) & X ( m + 1 ) & … & X ( {N}_{s} )\end{matrix} ] .$$

Singular value decomposition is performed on the Hankel matrix,

H=UΣV^{T},

wherein U and V are orthogonal matrixes which are a left singular matrix and a right singular matrix respectively,

Σ=diag(σ₁,σ₂, . . . ,σᵣ,

σ₁ is the singular values of the matrix H, and r is a number of the singular values.

S422: performing singular value decomposition on the Hankel matrix, and sorting the obtained singular values in a descending order.

S423: drawing an i-o; singular value graph after descending sorting, and acquiring an abscissa I corresponding to a starting point of a line segment with a maximum slope in the i−σ singular value graph, wherein σ₁ represents the ith singular value after descending sorting.

S424: determining the number P of the variation modal components according to

I=2P.

It should be noted that when the number of the decomposed variation modal components is optimal, a residual after the variation modal decomposition is a noise signal. Therefore, selection of the number of the variation modal components determines a noise characterization capability of the decomposed signal, and although an optimization algorithm in the prior art has a stronger global search capability, the optimization algorithm has a longer iteration process and a large calculation amount, cannot meet a real-time performance of recognition of the SSVEP electroencephalogram signal, and is prone to fall into local optimization. Therefore, in the present embodiment, the singular value is proposed to obtain the number of the variation modal components.

Since large singular values correspond to main information of the signals, large singular values represent valid signals with large frequency spectra and strong energy, and small singular values represent noise signals with small frequency spectra and weak energy, i.e., invalid signals. Therefore, the singular values are arranged in a descending order, a demarcation point (i.e., a position with the maximum slope) of the large singular value and the small singular value is found out according to the variation trend of the singular values, and the demarcation point can separate the valid signal from the invalid signal and is a point with the better number of the variation modal components.

The S43 specifically includes:

S431: calculating an analytic signal of each variation modal component by using Hilbert transformation;

$${u}_{i}^{+} ( t ) = ( δ ( t ) + \frac{j}{π t} ) * {u}_{i} ( t )$$

wherein uᵢ(t) is the ith variation modal component, uᵢ⁺(t) represents the analytic signal of the ith variation modal component, δ(t) represents an impulse function, j is an imaginary unit, and * represents a convolution operation.

S432: estimating a center frequency of each analytic signal, and shifting the analytic signal to a baseband by using a frequency shift operation;

$${u}_{i}^{+} ( t ) {e}^{- j {ω}_{i} t} = [ ( δ ( t ) + \frac{j}{π t} ) * {u}_{i} ( t ) ] {e}^{- j {ω}_{i} t}$$

wherein ωᵢ is the center frequency of the analytic signal of the ith variation modal component, and e^{-jωit} is an exponential correction term.

S433: calculating a gradient square norm of the analytic signal shifted to the baseband to carry out Gaussian smoothing, so as to estimate a bandwidth of each variation modal component and construct a constrained variation model;

$$\{ \begin{matrix}{min}_{\{ {u}_{i} ( t ) \} \{ {ω}_{i} ( t ) \}} \{ {\sum }_{i = 1}^{P} {‖ {\partial }_{t} {u}_{i}^{+} ( t ) {e}^{- j {ω}_{i} t} ‖}_{2}^{2} \} \\ s . t {Σ}_{i = 1}^{P} {u}_{i} ( t ) = X ( t )\end{matrix} \}$$

wherein X (t) is the second electroencephalogram signal, P is the total number of the variation modal components, and ∂ₜ represents solving of a partial derivative of t.

S434: converting the constrained variation model into an unconstrained variation model by adopting a secondary penalty factor and a Lagrange operator;

$$L ( \{ {u}_{i} ( t ) \} , \{ {ω}_{i} ( t ) \} , λ ) = {δΣ}_{i = 1}^{P} {‖ [ ( δ ( t ) + \frac{j}{π t} ) * {u}_{i} ( t ) ] {e}^{- j {ω}_{i} t} ‖}_{2}^{2} + {‖ X ( t ) - {Σ}_{i = 1}^{P} {u}_{i} ( t ) ‖}_{2}^{2} + 〈 λ ( t ) , X ( t ) - {Σ}_{i = 1}^{P} {u}_{i} ( t ) 〉$$

wherein a is the secondary penalty factor, λ(t) is the Lagrange operator, and < > represents an inner product operation.

S435: solving the unconstrained variation model by adopting the alternating direction multiplier algorithm, alternately updating the variation modal component, the center frequency and the Lagrange operator in the unconstrained variation model, and optimizing the variation modal component to obtain an optimized variational modal component.

S436: when the optimized variation modal components meet a judgment precision condition, finishing iteration and outputting each variation modal component.

As the judgment precision condition, when

$${Σ}_{i = 1}^{P} \frac{{‖ {u}_{i}^{n + 1} ( t ) - {u}_{i}^{n} ( t ) ‖}_{2}^{2}}{{‖ {u}_{i}^{n} ( t ) ‖}_{2}^{2}} < ε$$

is satisfied, each variation modal component is output, and otherwise, the process returns to continue iteration. uᵢⁿ(t) is an nth iteration value of the ith variation modal component, uᵢⁿ⁺¹(t) is an (n+1)th iteration value of the ith variation modal component, and ε>0 is preset judgment precision.

The influences of irrelevant brain activities and artifacts (the noise, namely the invalid signals) are reduced by extracting the valid variation modal components in the multi-channel SSVEP electroencephalogram signal. At the end of each iteration step, new variation modal components are obtained from the optimized variation modal components, and have different frequencies and amplitudes to capture different components of the signal. When the variation modal component reaches certain stability (meets the judgment precision), the iteration is terminated.

It should be noted that when the number of the decomposed variation modal components is optimal, the residual after the variation modal decomposition is noise. Therefore, in the prior art, a number of variation modal decomposition times of a variation modal decomposition algorithm is determined to optimally decompose the signal as far as possible; for example, the number of the optimal variation modal components is determined through a cross correlation coefficient of a signal superposing the residual and the variation modal component, but only qualitative analysis is realized, the decomposed residual cannot be the whole noise, other variation modal components also contain a small number of noise signals, and meanwhile, the residual variation modal components are removed through a manually set standard, and then, through an inverse operation of the algorithm, a denoised signal is reconstructed, and the removed residual components may contain valid signals, so that the electroencephalogram signal is deformed to different degrees due to direct deletion of the components, and the unremoved parts may also contain noise parts, and therefore, the obtained signal still contains noise. Considering that attention cannot be paid to the noise signal quantitatively to reduce the influence of the noise signal to the maximum extent, in the present invention, different weights are assigned to different variation modal components, thus improving a proportion of the valid components and reducing a proportion of the invalid components. The process specifically includes:

adopting relative entropy of the variation modal component and the second electroencephalogram signal as a fitness function of the sparrow search algorithm; the larger a value of the fitness function of the variation modal component, the larger the weight.

The relative entropy is a measure of asymmetry of a difference between two probability distributions. In the present invention, the difference between each variation modal component and the original non-decomposed signal is measured by using the relative entropy. It should be noted that the smaller the relative entropy of the component and the original signal is, the closer the component and the original signal are. A noise component is much smaller than a real signal part in the collected SSVEP signal, so that a component with a small difference from the original signal is recorded as a valid component and endowed with a larger weight; a component with a large difference from the original signal is recorded as an invalid component, and the weight of the invalid component is reduced. The weighting is carried out on the weight through the sparrow search algorithm, and then, the signal is reconstructed, so that a valid component proportion in the signal can be improved, and an invalid component proportion can be reduced, thereby achieving an artifact removing effect.

Specifically, the fitness function is

f=−[ϑ(uᵢ(t),Xt))+ϑ(X(t),uᵢ(t))]

wherein

$$ϑ ( {u}_{i} ( t ) , X ( t ) ) = \sum {u}_{i} ( t ) log \frac{{u}_{i} ( t )}{X ( t )} , ϑ ( X ( t ) , {u}_{i} ( t ) ) = \sum X ( t ) log \frac{X ( t )}{{u}_{i} ( t )}$$

represents the relative entropy, X (t) is the second electroencephalogram signal, and uᵢ(t) is the ith variation modal component.

Specifically, the sparrow search algorithm includes the following steps:
S441: initializing a population, an iteration time number and a proportion of finders, followers and guardians based on a weight range of the variation modal component, and randomly generating initial positions of sparrows, each sparrow representing one weight of one variation modal component;
S442: randomly selecting a target point for each sparrow at a current position, moving the sparrow towards the target point, and calculating a fitness function value of each sparrow based on the relative entropy of the variation modal component and the second electroencephalogram signal;
S443: updating position information of the finders, the followers and the guardians; and
S444: judging whether an iteration stopping condition is met, and if yes, outputting the fitness function values of all the sparrows at present to obtain the weights of the variation modal components; otherwise, returning to step S442.

The weight

W_{i,p}=[W_{i,1},W_{i,2}, . . . ,W_{i,p}]

of each variation modal component (IMF) is obtained through the above steps to obtain the weighted electroencephalogram signal:

Xᵢ=Wᵢ₁×IMFᵢ₁+Wᵢ₂×IMFᵢ₂+ . . . +W_{iP}×IMF_{iP},

wherein Xᵢ represents the electroencephalogram signal with the weighted and reconstructed ith channel, P is the total number of the variation modal components, and W_{iP} is different weights.

The S5 specifically includes: calculating a Pearson coefficient of the third electroencephalogram signal and an average signal of the third electroencephalogram signal and a Pearson coefficient of the third electroencephalogram signal and the reference signal by adopting a typical correlation analysis method as correlation coefficients, and taking a frequency corresponding to the maximum correlation coefficient as the evoking stimulus frequency of the third electroencephalogram signal.

Between the two signals of each pair, two weight vectors are created by using the typical correlation analysis method to allow correlation between the two weight vectors and a linear combination of the corresponding signals to be largest, the weight vectors are used as the spatial filters for filtering the two signals, and the Pearson coefficient between the filtered signals is calculated. The plurality of Pearson coefficients obtained between a plurality of pairs of signals are weighted, squared and then summed to obtain the correlation coefficient.

The calculating a Pearson coefficient of the third electroencephalogram signal and an average signal of the third electroencephalogram signal and a Pearson coefficient of the third electroencephalogram signal and the reference signal by adopting a typical correlation analysis method specifically includes:
calculating a spatial filter of the third electroencephalogram signal and the reference signal by adopting the typical correlation analysis method to obtain a first weight vector ω_{X}_{₁}, and solving a first Pearson coefficient ρ₁ of the third electroencephalogram signal X and the reference signal Y_{f} based on the first weight vector;

$${ρ}_{1} = ρ ( {X}^{T} {ω}_{{X}_{1}} ( X , {Y}_{f} ) , {Y}_{f}^{T} {ω}_{{X}_{1}} ( X , {Y}_{f} ) )$$

ρ( ) representing the calculation of the Pearson coefficient;
calculating a spatial filter of the third electroencephalogram signal and the average signal of the third electroencephalogram signal by adopting the typical correlation analysis method to obtain a second weight vector ω_{X}_{₂}, and solving a second Pearson coefficient of the third electroencephalogram signal X and the average signal X̅ of the third electroencephalogram signal based on the second weight vector;

$${ρ}_{2} = ρ ( {X}^{T} {ω}_{{X}_{2}} ( X , {\overline{X}}^{T} ) , {\overline{X}}^{T} {ω}_{{X}_{2}} ( X , {\overline{X}}^{T} ) )$$

the average signal of the third electroencephalogram signal being a mean of the third electroencephalogram signal under Nₜ trials of the same stimulus.

The correlation coefficient is therefore:

Rᵢ=sign(ρ₁ⁱ)·(ρ₁ⁱ)²+sign(ρ₂ⁱ)·(ρ₂ⁱ)²

Rᵢ represents a correlation coefficient of the ith stimulus and the reference signal, ρ₁ⁱ represents the first Pearson coefficient of the third electroencephalogram signal under the ith stimulus, ρ₂ⁱ represents the second Pearson coefficient of the third electroencephalogram signal under the ith stimulus, and sign ( ) is a sign function and used for representing positive or negative correlation.

The evoking stimulus frequency of the third electroencephalogram signal is

$$f = {arg max}_{i} {R}_{i} ,$$

wherein

i=1,2, . . . ,N_{f}

represents a stimulus target number.

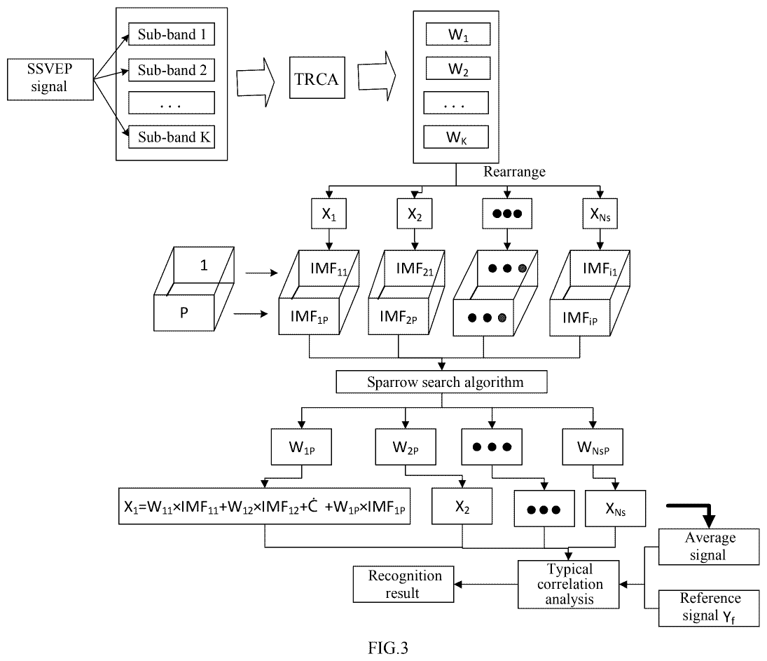

An overall framework of an intelligent ward round process based on the SSVEP electroencephalogram signal in the present invention is shown in FIG. 3.

In the present embodiment, before the correlation coefficient is determined, the influences of the noise and the artifacts are reduced by performing multiple decomposition and reconstruction on the SSVEP signal and endowing the valid component with a larger weight, so that the signal-to-noise ratio of the obtained signal is high, and therefore, self-feature information is extracted by an average experimental signal, individual and trial influences are reduced, and an average feature has higher accuracy and robustness; frequency recognition accuracy of the signal under a high signal-to-noise ratio can be effectively ensured in combination with the correlation coefficient calculated for the electroencephalogram signal and the reference signal.

Based on the assumption that the filters of different targets in the same frequency band are similar to each other, the space domain filters obtained for different stimulus targets are integrated and combined for target recognition, so that a performance of space domain filtering can be remarkably improved.

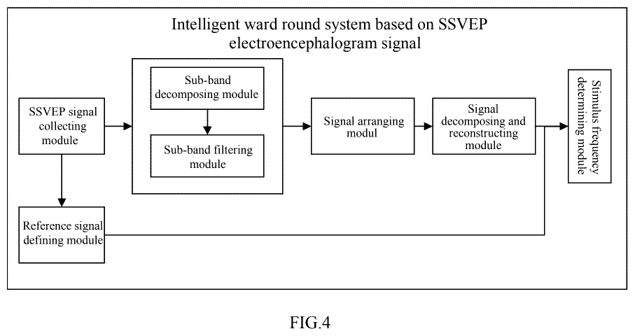

The present invention further provides an intelligent ward round device based on an SSVEP electroencephalogram signal, as shown in FIG. 4, including:

an SSVEP signal collecting module configured to collect a first electroencephalogram signal of a patient under a stimulus of a current visual stimulus source;

a reference signal defining module configured to define a reference signal according to a visual stimulus frequency for evoking the first electroencephalogram signal;

a sub-band decomposing module configured to decompose the first electroencephalogram signal into sub-band components distributed in different frequency ranges;

a sub-band filtering module configured to calculate differences of the sub-band components, a difference of the reference signal and differences between the sub-band components and the reference signal, and acquire a total spatial filter of the sub-band components to filter the sub-band components;

a signal arranging module configured to rearrange the sub-band components processed by the total spatial filter into a second electroencephalogram signal;

a signal decomposing and reconstructing module configured to acquire a plurality of variation modal components of the second electroencephalogram signal according to variation modal decomposition, and optimize weights of the variation modal components under each channel to reconstruct an electroencephalogram signal to obtain a third electroencephalogram signal; and

a stimulus frequency determining module configured to determine a correlation coefficient based on own features of the third electroencephalogram signal and the reference signal to obtain an evoking stimulus frequency of the third electroencephalogram signal for determining an intention of the patient during an intelligent ward round.

For the specific limitations of the intelligent ward rounds device based on SSVEP electroencephalogram signals, please refer to the above limitation of the intelligent ward rounds based on SSVEP electroencephalogram signals, which will not be repeated here.

The present invention is not limited to the above-described embodiments, and various modifications made by those skilled in the art without creative efforts from the above-described conception fall within the protection scope of the present invention.

What is claimed is:

1. An intelligent ward round method based on an SSVEP electroencephalogram signal, the interactive terminal is used to present a visual stimulus source that includes a plurality of candidate intention options to the patient, the acquisition terminal is used to collect the patient's electroencephalogram signal, the electroencephalogram signal obtained by the acquisition terminal is transmitted to the interactive terminal for electroencephalogram signal recognition, the patient's expression intention is determined, and the expression intentions comprise disease intentions, privacy intentions, psychological intentions, physiological intentions, environmental intentions, and safety intentions, and the intelligent ward round is completed; the method comprising:
S1: acquiring a preprocessed electroencephalogram signal of a patient under a stimulus of a current visual stimulus source, recording the preprocessed electroencephalogram signal as a first electroencephalogram signal, and defining a reference signal according to a visual stimulus frequency for evoking the first electroencephalogram signal;
S2: decomposing the first electroencephalogram signal into sub-band components distributed in different frequency ranges, calculating differences of the sub-band components, a difference of the reference signal and differences between the sub-band components and the reference signal, and acquiring a total spatial filter of the sub-band components;
S3: processing each sub-band component by the total spatial filter, rearranging the sub-band components into a new multi-channel signal, and recording the new multi-channel signal as a second electroencephalogram signal;
S4: acquiring a plurality of variation modal components of the second electroencephalogram signal by adopting variation modal decomposition, and optimizing weights of the variation modal components under each channel to reconstruct an electroencephalogram signal to obtain a third electroencephalogram signal; and
S5: determining a correlation coefficient based on own features of the third electroencephalogram signal and the reference signal to obtain an evoking stimulus frequency of the third electroencephalogram signal, and determining an intention of the patient based on the evoking stimulus frequency to complete an intelligent ward round.

2. The intelligent ward round method based on an SSVEP electroencephalogram signal according to claim 1, wherein the preprocessing in the step S₁ comprises:
performing signal amplification, filtering and analog-to-digital conversion on the acquired electroencephalogram signal of the patient under a rhythmic visual stimulus in an SSVEP paradigm;
the filtering comprises passing the electroencephalogram signal through a 50 Hz notch filter and a 3-40 Hz Butterworth filter in sequence to obtain the first electroencephalogram signal.

3. The intelligent ward round method based on an SSVEP electroencephalogram signal according to claim 1, wherein the defining a reference signal according to a visual stimulus frequency for evoking the first electroencephalogram signal in the step S1 comprises:
constructing sine and cosine periodic signals as the reference signal based on the visual stimulus frequency for evoking the first electroencephalogram signal, a frequency of the reference signal being the same as the visual stimulus frequency for evoking the first electroencephalogram signal or being a multiple of the visual stimulus frequency for evoking the first electroencephalogram signal.

4. The intelligent ward round method based on an SSVEP electroencephalogram signal according to claim 3, wherein the decomposing the first electroencephalogram signal into sub-band components distributed in different frequency ranges in the step S2 specifically comprises: constructing a decomposition filter bank, and decomposing the first electroencephalogram signal into a plurality of frequency bands by using the decomposition filter bank to obtain the sub-band components of the first electroencephalogram signal, specifically comprising:
S21: determining a number K of the sub-band components based on a frequency range of the first electroencephalogram signal, the decomposition filter bank comprising a plurality of band-pass filters, and each band-pass filter being configured to extract information of one specific frequency range of the first electroencephalogram signal to obtain one sub-band component;
S22: calculating a center frequency of each band-pass filter, the center frequency being selected based on an equidistant frequency between the frequency ranges of the first electroencephalogram signal;
S23: establishing one band-pass filter for each center frequency, the transfer function of the band-pass filter being:
$$H ( z ) = \frac{z - \frac{1}{2} cos ( 2 π {f}_{c} )}{z - cos ( 2 π {f}_{c} )}$$
wherein f_{c} denotes the center frequency, and z denotes a complex variable;
S24: decomposing the first electroencephalogram signal into K sections in a frequency domain through the decomposition filter bank, each section being one sub-band component to obtain K sub-band components of the first electroencephalogram signal, and the sub-band components being distributed in different frequency ranges.

5. The intelligent ward round method based on an SSVEP electroencephalogram signal according to claim 4, wherein the step S2 of calculating differences of the sub-band components, a difference of the reference signal and differences between the sub-band components and the reference signal, and acquiring a total spatial filter of the sub-band components specifically comprises:
maximizing a trial difference index of the same sub-band component in different trials under the same stimulus, a reference difference index between the same sub-band component and the reference signal in different trials under the same stimulus and a template difference index of the reference signal by using a TRCA method, acquiring the spatial filter of the sub-band component under the corresponding stimulus, and connecting all the spatial filters under each stimulus to obtain the total spatial filter of each sub-band.

6. The intelligent ward round method based on an SSVEP electroencephalogram signal according to claim 5, wherein the trial difference index, the reference difference index and the template difference index are measured through covariances, and specifically:
the trial difference index is a sum of the covariances of the same sub-band component in different trials under the same stimulus and is recorded as
S₁=Σ_{i,j}^{Nt} Cov(x_{K}ⁱ,x_{K}ⁱ)
wherein i and j represent different trials under the same stimulus, i≠j, K represents a Kth sub-band component, Nₜ represents a number of the trials, and Cov ( ) represents the covariance;
the reference difference index comprises a sum S₂₁ of covariances between the same sub-band component in the trials under the same stimulus and a sine periodic signal in the reference signal and a sum S₂₂ of covariances between the same sub-band component in the trials under the same stimulus and a cosine periodic signal in the reference signal;
wherein
S₂₁=Σᵢ^{Nt} Cov(x_{K}ⁱ,Y_{f}^{s}),
and Y_{f}^{s} is the sine periodic signal in the reference signal;
S₂₂=Σᵢ^{Nt} Cov(x_{K}ⁱ,Y_{f}^{c}),
and Y_{f}^{c} is the cosine periodic signal in the reference signal;
x_{K}ⁱ represents a sub-component of the Kth sub-band component at an ith trial;
the template difference index is a sum of covariances between the sine and cosine periodic signals,
the template difference index is recorded as
S₃=Σᵢ^{Nt} Cov(Y_{f}^{s},Y_{f}^{c}),
wherein Y_{f}^{s} and Y_{f}^{c} are the sine periodic signal and the cosine periodic signal in the reference signal.

7. The intelligent ward round method based on an SSVEP electroencephalogram signal according to claim 6, wherein the obtaining a total spatial filter of the sub-bands specifically comprises:
constructing a difference matrix
$$S = [ \begin{matrix}{S}_{1} & {S}_{21} \\ {S}_{22} & {S}_{3}\end{matrix} ]$$
based on the trial difference index, the reference difference index and the template difference index;
solving
Wₙ^{K}=argmax(Wₙ^{K})^{T}Sₙ^{K}Wₙ^{K}
to obtain a weight of the spatial filter, and constructing the spatial filter under the corresponding stimulus; wherein Wₙ^{K} is the weight of the spatial filter, and Sₙ^{K} is the difference matrix of the Kth sub-band component under the nth stimulus; and
obtaining the final spatial filter for the K sub-band components by connecting the weights of all the spatial filters at each stimulus:
W^{K}=[W₁^{K},W₂^{K}, . . . W_{N}_{_{f}}^{K}],
wherein N_{f} represents a total number of the stimuli.

8. The intelligent ward round method based on an SSVEP electroencephalogram signal according to claim 1, wherein the S3 of processing each sub-band component by the total spatial filter, rearranging the sub-band components into a new multi-channel signal, and recording the new multi-channel signal as a second electroencephalogram signal specifically comprises:
vertically arranging and organizing the first electroencephalogram signal subjected to TRCA space domain filtering into new channels along the y axis according to a sequence of the N_{f} spatial filters; and
recombining the K sub-band components along the z axis in each new channel to obtain a new signal formed by rearrangement, and recording the new signal as the second electroencephalogram signal.

9. The intelligent ward round method based on an SSVEP electroencephalogram signal according to claim 1, wherein the S4 of acquiring a plurality of variation modal components of the second electroencephalogram signal by adopting variation modal decomposition, and optimizing weights of the variation modal components under each channel to reconstruct an electroencephalogram signal to obtain a third electroencephalogram signal comprises:
S41: representing the second electroencephalogram signal as a sum of the plurality of variation modal components to carry out singular value decomposition on the second electroencephalogram signal;
S42: determining a number P of the variation modal components based on a variation trend of singular values;
S43: constructing a variation model based on the number P of the variation modal components, and solving the variation model by adopting an alternating direction multiplier algorithm to acquire the plurality of variation modal components of the second electroencephalogram signal;
S44: weighting the variation modal components of each frequency band by adopting a sparrow search algorithm, weights of the variation modal components being determined according to a fitness value of the sparrow search algorithm; and
S45: reconstructing the electroencephalogram signal under each channel based on the weights of the variation modal components to obtain the third electroencephalogram signal.

10. The intelligent ward round method based on an SSVEP electroencephalogram signal according to claim 9, wherein the S42 of determining a number P of the variation modal components based on a variation trend of singular values comprises:
S421: constructing an m×n order Hankel matrix based on the variation modal components and the channel number of the second electroencephalogram signal, wherein
$$m = [ \frac{{N}_{s}}{2} + 1 ] , n = {N}_{s} - m + 1 ,$$
Nₛ is the channel number, and [ ] represents upward rounding;
S422: performing singular value decomposition on the Hankel matrix, and sorting the obtained singular values in a descending order;
S423: drawing an i−σ₁ singular value graph after descending sorting, and acquiring an abscissa I corresponding to a starting point of a line segment with a maximum slope in the i−σ singular value graph, wherein σᵢ represents the ith singular value after descending sorting; and
S424: determining the number P of the variation modal components according to I=2P.

11. The intelligent ward round method based on an SSVEP electroencephalogram signal according to claim 10, wherein the S43 of constructing a variation model based on the number P of the variation modal components, and solving the variation model by adopting an alternating direction multiplier algorithm to acquire the plurality of variation modal components of the second electroencephalogram signal comprises:
S431: calculating an analytic signal of each variation modal component by using Hilbert transformation;
S433: calculating a gradient square norm of the analytic signal shifted to the baseband to carry out Gaussian smoothing, so as to estimate a bandwidth of each variation modal component and construct a constrained variation model;
S434: converting the constrained variation model into an unconstrained variation model by adopting a secondary penalty factor and a Lagrange operator;
S435: solving the unconstrained variation model by adopting the alternating direction multiplier algorithm, alternately updating the variation modal component, the center frequency and the Lagrange operator in the unconstrained variation model, and optimizing the variation modal component to obtain an optimized variational modal component; and
S436: when the optimized variation modal components meet a judgment precision condition, finishing iteration and outputting each variation modal component.

12. The intelligent ward round method based on an SSVEP electroencephalogram signal according to claim 9, wherein the S44 of weighting the variation modal components of each frequency band by adopting a sparrow search algorithm, weights of the variation modal components being determined according to a fitness function value of the sparrow search algorithm comprises:
adopting relative entropy of the variation modal component and the second electroencephalogram signal as a fitness function of the sparrow search algorithm;
the larger the value of the fitness function of the variation modal component, the larger the weight.

13. The intelligent ward round method based on an SSVEP electroencephalogram signal according to claim 1, wherein the S5 comprises:
calculating a Pearson coefficient of the third electroencephalogram signal and an average signal of the third electroencephalogram signal and a Pearson coefficient of the third electroencephalogram signal and the reference signal by adopting a typical correlation analysis method as correlation coefficients, and taking a frequency corresponding to the maximum correlation coefficient as the evoking stimulus frequency of the third electroencephalogram signal.

14. The intelligent ward round method based on an SSVEP electroencephalogram signal according to claim 13, wherein the calculating a Pearson coefficient of the third electroencephalogram signal and an average signal of the third electroencephalogram signal and a Pearson coefficient of the third electroencephalogram signal and the reference signal by adopting a typical correlation analysis method specifically comprises:
calculating a spatial filter of the third electroencephalogram signal and the reference signal by adopting the typical correlation analysis method to obtain a first weight vector ω_{X}_{₁}, and solving a first Pearson coefficient ρ₁ of the third electroencephalogram signal X and the reference signal Y_{f} based on the first weight vector; and
calculating a spatial filter of the third electroencephalogram signal and the average signal of the third electroencephalogram signal by adopting the typical correlation analysis method to obtain a second weight vector ω_{X}_{₂}, and solving a second Pearson coefficient of the third electroencephalogram signal X and the average signal X̅ of the third electroencephalogram signal based on the second weight vector.

15. The intelligent ward round method based on an SSVEP electroencephalogram signal according to claim 14, wherein the correlation coefficient specifically is:
$${R}_{i} = sign ( {ρ}_{1}^{i} ) \cdot {( {ρ}_{1}^{i} )}^{2} + sign ( {ρ}_{2}^{i} ) \cdot {( {ρ}_{2}^{i} )}^{2}$$
wherein Rᵢ represents a correlation coefficient of the ith stimulus and the reference signal, ρ₁ⁱ represents the first Pearson coefficient of the third electroencephalogram signal under the ith stimulus, ρ₂ⁱ represents the second Pearson coefficient of the third electroencephalogram signal under the ith stimulus, and sign ( ) is a sign function and used for representing positive or negative correlation, the evoking stimulus frequency of the third electroencephalogram signal is
$$f = {arg max}_{i} {R}_{i} ,$$
wherein i=1, 2, . . . , N_{f} represents a stimulus target number.
